(19) United States
(12) Patent Application Publication
(10) Pub. No.: US 2010/0143942 A1
(43) Pub. Date: Jun. 10, 2010

(54) METHOD OF DETECTION

(75) Inventors: Gerard W. Canters, Leiden (NL); Gerhild Zauner, Leiden (NL); Armand W.J.W. Tepper, Leiden (NL); Luigi Bubacco, Padua (IT); Thijs J. Aartsma, Leiden (NL)

Correspondence Address: WHITHAM, CURTIS & CHRISTOFFERSON & COOK, P.C., 11491 SUNSET HILLS ROAD, SUITE 340, RESTON, VA, 20190, US

(73) Assignees: LEIDEN UNIVERSITY, Leiden (NL); UNIVERSITY OF PADUA, Padua (NL)

(21) Appl. No.: 12/442,262

(22) PCT Filed: Sep. 21, 2006
(86) PCT No.: PCT/EP2007/060064

§ 371 Date: Jan. 27, 2010

(30) Foreign Application Priority Data
Sep. 21, 2006 (EP) 06121062.1

(51) Int. Cl. G01N 33/53; G01N 21/64; C12M 1/00; C07K 14/00; C12N 9/00; C12N 9/02

(52) U.S. Cl. 435/7.8; 436/501; 422/82.08; 435/288.7; 530/350; 435/183; 435/189

(57) ABSTRACT

A method of detection of an analyte in which (i) a protein comprising a moiety capable of binding the analyte and a fluorescent label is contacted with a medium suspected of containing the analyte; (ii) the analyte, if present, binds to the moiety; (iii) the protein is subjected to incident radiation to excite the protein and induce intrinsic emission therefrom; whereby the intrinsic emission from the protein is converted through Fluorescence Resonance Energy Transfer (FRET) into emission from the fluorescent label and the amount of said FRET is affected by the binding of the analyte to the moiety; and, (iv) the emission from the fluorescent label is measured; whereby the level of emission from the fluorescent label is indicative of the presence of the analyte, and wherein the protein undergoes no substantial conformational charge during the method. A kit for carrying out the method of the invention and a protein are also provided.

The present invention relates to a fluorescence resonance energy transfer (FRET) based method of detection of an analyte using a protein capable of binding the analyte. The present invention has particular utility in oxygen sensing.

FRET is based on a distance-dependent interaction between the electronic excited states of two dye molecules in which excitation is transferred from a donor molecule to an acceptor molecule without the emission of a photon. This process is known as Förster energy transfer. The efficiency of FRET is dependent on the inverse sixth power of intermolecular separation [1], making it useful over distances comparable with the dimensions of biological macromolecules. When FRET is used as a contrast mechanism, colocalisation of proteins and other molecules can be imaged with spatial resolution beyond the limits of conventional optical microscopy [2].

In order for FRET to occur the donor and acceptor molecules must be in close proximity (typically 10-100 Å), the absorption spectrum of the acceptor must overlap with the fluorescence emission spectrum of the donor, and the donor and acceptor transition dipole vectors must be approximately parallel, or at least not orthogonal.

Existing methods of oxygen detection are based often on (bulky) solid state sensors, the prototype of which is the Clark electrode [3]. One of the Clark electrode's drawbacks is that it consumes oxygen during measurement, thereby changing the oxygen concentration in the sample. Other sensors are based on measuring the luminescence of an oxygen sensitive reporter molecule [4], usually a ruthenium compound. Such a compound has the drawback of being unstable in water and therefore requiring encapsulation in a hydrophobic matrix. Application of both types of sensors is further limited because of possible interference by other substances and because of relatively long response times.

We have described in a previous patent application, [5], how FRET can be used to monitor the activity of a donor-acceptor pair on a protein. In that application, a labelled protein comprising a fluorescent donor label and an energy acceptor moiety is subjected to incident radiation to excite the donor and the fluorescence emission of the donor is measured. The emission intensity varies with the level of quenching by the acceptor moiety, which typically depends upon its oxidation state.

Erker et al [6] have described how fluorescence labels may be used as sensors for oxygen binding of arthropod hemocyanins. FRET from fluorescent labels to oxygenated active sites quenches the emission of the labels by roughly 50% upon oxygenation of the protein.

As detailed in Erker et al, proteins belonging to the “type-3 copper” protein family have well characterised oxygen-binding ability.

A type-3 copper site consists of two closely spaced copper ions each coordinated by three histidine residues. Molecular oxygen reversibly binds to the reduced and colourless [Cu(I)—Cu(I)] type-3 centre to yield the oxygenated [Cu(I)—O₂—Cu(I)] species in which oxygen is bound in a Cu₂ bridging ‘side-on’ geometry. The oxy form is characterized by an optical transition around 345 nm and a weak d-d transition at higher wavelength, around 570 nm. Oxygen binding can be followed spectroscopically, by measuring the absorption at 345 or 570 nm. Due to the relatively low extinction coefficients, however, this is an insensitive method to measure oxygen concentrations. Moreover, it is not selective.

A further method of oxygen detection has been devised based on tryptophan fluorescence. When excited by UV light (280-290 nm), proteins often exhibit a conspicuous fluorescence that originates from the aromatic residues phenylalanine, tyrosine and in particular tryptophan. In the case of type-3 Cu proteins, the emission of the tryptophan residues can be quenched by FRET to the 345 nm absorption band provided the protein is in the oxy-form. In the de-oxy form this band and, therefore, the quenching is absent. It follows that the tryptophan fluorescence is a reporter of the amount of oxygen bound to the type-3 centres, which is a direct measure of the oxygen concentration in solution. By measuring the tryptophan fluorescence instead of the absorbance at 345 or 570 nm, oxygen concentrations may be obtained. However, the method is not selective: many compounds other than tryptophan residues in a protein are excited by UV light and start to fluoresce. Furthermore, the detection of near-UV emission is cumbersome in scattering (biological) media.

US 2002/0165364 describes fluorescent indicators including a binding protein moiety, a donor fluorescent protein moiety and an acceptor fluorescent protein moiety. The binding protein moiety has an analyte-binding region which binds an analyte and causes the indicator to change conformation upon exposure to the analyte. Changes in fluorescence emission are due to protein conformational changes. Similarly, [17] describes a glucose sensor which involves measuring changes in FRET which are caused by changes in donor-acceptor distance when the analyte (glucose) binds.

In accordance with the first aspect of the present invention we provide a method of detection of an analyte in which,

(i) a protein comprising a moiety capable of binding the analyte and a fluorescent label is contacted with a medium suspected of containing the analyte;

(ii) the analyte, if present, binds to the moiety;

(iii) the protein is subjected to incident radiation to excite the protein and induce intrinsic emission therefrom;

whereby the intrinsic emission from the protein is converted through Fluorescence Resonance Energy Transfer (FRET) into emission from the fluorescent label and the amount of said FRET is affected by the binding of the analyte to the moiety; and,

(iv) the emission from the fluorescent label is measured;

whereby the level of emission from the fluorescent label is indicative of the presence of the analyte, and wherein the protein undergoes no substantial conformational change during the method.

By a proper choice of the fluorescent label the present invention provides both a sensitive and selective method for the detection of an analyte. Since FRET efficiency depends on the inverse sixth power of the distance between donor and acceptor, the label (acceptor) will only communicate with amino acid residues (donors) in close proximity, i.e., residues located on the same protein molecule as the label. Energy transfer from other molecules is not efficient enough to compete with the intra-molecular energy transfer to the label. The advantage of measuring the sensitised fluorescence is that the signal is free from background noise. Since the label can be chosen so that the emission occurs in the visible range of the spectrum where there is no interference from other emitting species in the solution, the method is selective. Moreover, depending on the efficiency of energy transfer, there may also be a gain in quantum yield of the fluorescence when comparing the sensitized fluorescence with the intrinsic fluorescence of the protein. This also helps to increase the sensitivity of the method proposed herein.

By no substantial conformational change, we mean that analyte binding does not cause a change in donor (i.e. source of intrinsic emission) and acceptor (i.e. fluorescent label) distance such as to affect the FRET between them. Analyte binding should also not significally alter the donor-moiety distance, since the moiety may also be a FRET acceptor, for instance when analyte binds. Typically, analyte binding affects the donor-acceptor distance by less than 10%, preferably less than 5%, most preferably less than 1%.

In one embodiment of the invention the moiety binds reversibly to the analyte to allow the protein to be recycled and used in a subsequent method of detection. Thus, the analyte is chemically identical before and after binding.

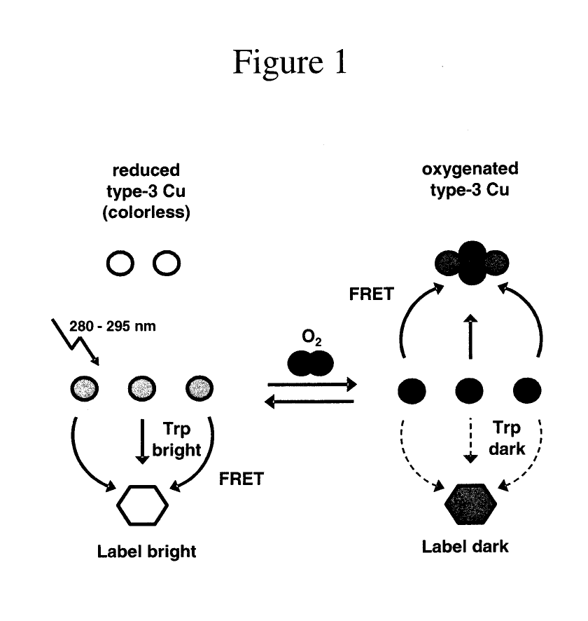

Typically, the binding of the analyte to the moiety reduces the amount of intrinsic emission from the protein converted through FRET into emission from the label. The binding of the analyte may allow a second FRET channel to be opened up to the moiety, consequently reducing the energy channelled to the label and inducing a drop in label fluorescence. For this to occur, the absorption spectrum of the moiety when bound to the analyte should overlap with the intrinsic emission from the protein. The concept is illustrated further in FIG. 1.

Typically, when no analyte is bound, no FRET occurs to the moiety.

As detailed above, when excited by UV light, proteins often exhibit a conspicuous fluorescence that originates from aromatic residues. Typically, this fluorescence, or “intrinsic emission” from the protein is due to the tryptophan residues in the protein. Alternatively, phenylalanine or tyrosine residues, or an organic cofactor may fluoresce.

The emission from the fluorescent label is typically not “all or nothing” (within a bulk solution) and may vary proportionally with the concentration of analyte in the medium suspected of containing the analyte. This advantageously allows the concentration of the analyte in the medium to be determined. When observing single molecules, the fluorescence is typically on or off (or high or low). The concentration of the analyte in the single molecule case is then reflected by the average number of the on and off periods per unit time.

It is envisaged that the invention will have utility wherein the protein is an enzyme, preferably a redox enzyme. The analyte may be a (co-)substrate, inhibitor or cofactor of the enzyme. A (co-)substrate in this specification means one of two or more substrates of an enzyme. The analyte is typically a molecule which binds to the protein and causes a change in the intrinsic fluorescence of the protein. The analyte typically associates with the moiety through non-covalent interactions. The dissociation constant for an analyte may vary widely, spanning a range from nM to mM.

The analyte may be converted to another species by the enzyme, or alternatively may bind reversibly, as in the case of some enzyme inhibitors.

The moiety is capable of binding the analyte. Typically the moiety is a chromophore, the absorption of which changes as a result of enzymatic activity, or analyte binding. Enzymes typically contain metal ions, metal ion complexes comprising two or more metal ions (preferably transition metal ions) and organic cofactors (such as flavin) for binding of external molecules. Copper, iron and nickel ions are particularly preferred metal ions. Suitable organic cofactors include orthoquinone and pyridoxal-5-phosphate. Any of these may constitute the moiety of the protein used in the present invention.

A suitable moiety is Cu₂, as in tyrosinase. Alternatively, the moiety may be Cu₃, as in laccase. An analyte (for example oxygen) may reversibly bind to Cu₂. Alternatively, the analyte may bind to the moiety and be converted to product. When O₂ binds to a Cu₃ centre, for example, the centre is (partly) reduced and the O₂ is converted to peroxide or water. An inhibitor of an enzyme comprising a Cu₃ centre, on the other hand, may bind reversibly to the centre.

Preferably, the protein is an oxygen carrier, oxygenase enzyme or an oxidase. The oxygen carrier may be hemocyanin (Hc) from an anthropod or mollusc, or alternatively haemerythrin. Suitable oxygenases catalyse the hydroxylation of phenols and the oxidation of the diphenol products to the corresponding quinones (for example, tyrosinase, Ty). The oxygenase enzyme may be a monooxygenase. Suitable oxidases may convert o-diphenols to the corresponding quinones (for example, catechol oxidase, CO).

Other proteins which may have utility in the present invention include laccase, polyphenol oxidases, cytochrome P450 enzymes, hydrogenases and ureases.

The analyte is typically a gas under standard temperature and pressure, such as O₂, H₂, CO₂, CO, NO or N₂O. The preferred analyte which is detected in the method according to the first aspect of this invention is oxygen. In this preferred embodiment, the protein is typically a redox enzyme and catalyses the oxidation of a substrate using oxygen bound to the protein. When the protein is tyrosinase, for example, the analyte is oxygen and the substrate is a monophenol or an ortho-diphenol, for example, tyrosine. The redox enzyme preferably has a binding site for the substrate, which may be the moiety which binds the analyte, or alternatively a different moiety which has suitable properties for binding the substrate.

The analyte may alternatively be hydrogen. A suitable protein for detecting hydrogen is hydrogenase.

Other suitable analyte-protein combinations include:

ATP—ArsA ATPase (an arsenicum transport protein);

O₂/CO—cytochrome P450

Glucose—apo glucose oxidase;

Ca²⁺—C-type mannose binding protein;

Decamethonium, edrophonium, ethopropazine, acetylcholine or choline—cholinesterase;

Ca²⁺ or Mg²⁺—Guanylyl Cyclase-activating Proteins; and

Inhaled anaesthetics (e.g. halothane)—G-protein coupled receptors.

In all of these examples, the intrinsic fluorescence of the protein, the tryptophan fluorescence, changes upon the binding of the analyte.

The method according to the present invention may advantageously be selective for a particular analyte. Hemocyanins, for example, have evolved to selectively bind O₂, while minimizing interactions with other (biological) compounds. A further advantage when detecting oxygen is that the contrast between label emission for the O₂-free and O₂-bound protein may exceed the contrast observed for the Trp fluorescence. Furthermore, protein response times down to the msec range may be obtained in solution, being limited only by the dissociation rate of O₂ from the protein (e.g. 300 s⁻¹ for S. a. Ty).

The method of detection according to the present invention may be used to monitor the presence and/or concentration of molecules other than the analyte which affect the binding of the analyte to the moiety. For example, since oxygen carrier molecules such as Hc evolved to respond to the physiological demands of an organism, molecules like lactate, uric acid or ions (e.g. H⁺) change the oxygen binding properties and, thus, (in in vivo measurements) the emission measurements in the method according to the invention. These molecules (e.g. “allosteric effectors”) typically bind at a position on the protein distant from the moiety and have a (typically small) effect on the structure of the protein. It has been found that some Hcs are more sensitive than others to allosteric effectors. The use of two different Hcs carrying different fluorescent labels in the same sample allows the determination of a fluorescence intensity ratio. If it is known how the effector changes the O₂ dissociation constant for the more ‘sensitive’ protein, the measurement of the fluorescence response of both proteins provides information regarding the concentration of both O₂ and the effector.

The present invention is not limited to the detection of one analyte. The method may be used to detect two or more analytes in a medium. In this embodiment, two proteins which bind different analytes and each comprising different fluorescent labels are used. The two proteins are preferably excitable at the same wavelength.

Alternatively, two or more proteins may be used in the method of the invention with different dissociation constants for the same analyte. This advantageously increases the concentration range of analyte that may be detected, and the accuracy of the concentration measurements.

In these embodiments, the proteins preferably comprise fluorescent labels that fluoresce at different wavelengths. This allows the proteins to be monitored independently, thereby increasing accuracy and reproducibility.

The method of the present invention may further comprise a step of relating the emission from the fluorescent label to substrate turnover. During a typical oxidation reaction, oxygen is consumed and therefore the emission from the labelled protein would increase when used in this embodiment of the method according to this invention. The rate of increase of emission can be correlated with oxygen (O₂) consumption and therefore also substrate turnover, according to the stoichiometric relationship between the substrate and O₂ in the reaction mechanism.

The protein used in the method of the present invention may be added to a biological sample before being subject to incident radiation. This may advantageously allow the metabolic rate of the biological sample to be determined. There is no need for macroscopic mechanical interfacing as with the O₂ measurement systems of the prior art. Proteins such as the type-3 copper proteins have evolved to selectively bind oxygen in a biological setting, with minimal interference from other compounds. Oxygen binds reversibly and no O₂ is consumed during the method.

Typically, the biological sample is from an animal or human, typically animal or human tissue, more typically muscle, nerve or brain tissue. Respiration occurring in the biological sample consumes oxygen (the analyte) resulting in an increase over time in fluorescence from the sample. The protein may be added to the sample either in vivo (for example by direct injection into the tissue) or ex vivo (the protein is added to tissue obtained from cell cultures or from tissue following extraction of the tissue from the animal or human body).

Alternatively, the gene encoding the protein may be inserted into a cell by standard genetic techniques. The gene should carry a small modification such that the protein, when expressed, may be labelled by a fluorescent label added to the extracellular medium. The fluorescent label is typically able to cross the cell membrane or wall to attach spontaneously to the protein at a predesignated place.

As oxygen metabolism is elementary to all O₂ respiring organisms, its quantification in biological samples is highly valuable. The O₂ consumption is a direct measure of metabolic rate, which in turn is a measure of the activity of living cells.

The metabolic rate is also an important parameter in drug-screening, i.e. the screening of large libraries of drug candidates using mass in vitro diagnostics. O₂ consumption is a parameter of cell viability (with or without drugs added) to interrogate drug candidates for toxicity properties.

Since the protein is a biomolecule, it is possible to couple the protein to a second biomolecule that acts as a recognition element and binds to a specific target. For example, the protein may be coupled to an antibody that specifically binds to a receptor only found on a specific cell type. In this embodiment, the sensor can be specifically targeted to certain cells in complex tissue in order to measure oxygen only at the sites of interest using fluorescence microscopy.

This method may be applied to monitor oxygen concentration [O₂] in different (microscopic) sample compartments by targeting Cu type-3 protein conjugates to specific locations in the sample. For instance, [O₂] could be monitored at the surface of living cells by conjugating the labelled Cu type-3 protein with an antibody specific for a certain surface epitope, while unconjugated Cu type-3 protein carrying a different label could be utilized to determine [O₂] in the solution matrix surrounding the cell. Combining this scheme with fluorescence microscopy methods would enable measurement of O₂ consumption at sub-cellular levels and millisecond time scales and to monitor, for example, metabolic activity.

For the method of the present invention to allow detection of an analyte, FRET must be possible between the fluorescent protein residues and the fluorescent label, i.e. the emission spectra of the fluorescent protein residues should overlap with the absorption spectrum of the fluorescent label and the residues and label must be in sufficiently close proximity for FRET to occur. In addition, the absorption spectrum of either the bound or unbound moiety of the protein must overlap with the emission spectra of the fluorescent protein residues to allow a change in emission from the fluorescent label when an analyte binds to the protein.

Preferably, the fluorescent label absorbs radiation in the wavelength range 330-450 nm, preferably most strongly at about 350 nm, and fluoresces in the visible range of the spectrum, i.e. 400-700 nm. Fluorescence of the label in the visible region of the spectrum advantageously reduces the interference or ‘noise’ from fluorescence of protein residues. Suitable fluorescent labels include Cy5, Atto390, Atto655, Alexa350 and Cy3.

The incident radiation should be suitable for exciting the protein to induce intrinsic fluorescence therefrom. Preferably, the incident radiation has a wavelength of 260-450 nm, more preferably 280-300 nm if Trp residues are to be excited.

The fluorescent label may be associated with the protein through any conventional means known in the art. Typically the fluorescent label is conjugated to a cysteine, lysine or arginine residue or the N-terminus of the protein, optionally through a linker, such as N-hydroxysuccinimide. The label position can be varied by replacing a solvent-exposed residue by a cysteine residue. A label can then be specifically attached to this residue using a sulfhydryl reactive label (usually containing a maleimide reactive group), as detailed in [5].

Typical moiety-fluorescent label distances are in the range 1-4 nm. This is typically around the Förster distance. However, for the present invention, the distance from the fluorescent label to the moiety is less important than the distance from the label to the source of endogenous fluorescence (usually the Trp residues). Proteins usually contain several Trp residues and these should be within the Förster distance of the moiety for a quenching effect to be observed.

The labelled protein may be present in solution in the method according to the first aspect of this invention, or alternatively be immobilised on a carrier. Preferably, the carrier is an electrode, particularly when the protein is a redox enzyme. Associating the labelled protein with electrodes allows direct electron transfer between the protein and the electrodes. This offers potentiostatic control over the redox state of the surface layer, and the possibility to perform scanning voltammetry while detecting the fluorescence intensity. When the labelled protein is in solution, electrodes may alternatively be contacted with the solution in which the protein is dissolved.

Alternatively, the labelled protein may be immobilised in microparticles of a sol-gel gas-permeable matrix. The matrix should allow diffusion of the analyte from the medium suspected of containing the analyte to the protein immobilised in the matrix. This method of immobilisation advantageously provides high concentrations of the protein molecule. This may be advantageous, for example, in in vivo confocal microscopy.

In accordance with the second aspect of this invention we provide a kit comprising a protein comprising a fluorescent label, an analyte, a radiation source for imposing incident radiation at a suitable wavelength for exciting the protein and inducing intrinsic emission therefrom, and a radiation detector capable of detecting the fluorescence emitted by the label, wherein the protein additionally comprises a moiety capable of binding the analyte and wherein the intrinsic emission from the protein may be converted through Fluorescence Resonance Energy Transfer (FRET) into emission from the fluorescent label, the amount of said FRET is affected by the binding of the analyte to the moiety, and wherein the binding of the analyte to the moiety does not induce a substantial conformational change in the protein.

The kit is suitable for carrying out the method of the present invention. All of the preferred features of the method with regard to the protein, moiety, analyte and fluorescent label are also applicable to the kit of the present invention.

Preferably the radiation source has a wavelength of 260-450 nm to excite the protein, preferably 280-300 nm if tryptophan residues are to be excited.

Typically, the protein is an enzyme, preferably a redox enzyme. The analyte may be a substrate, cofactor or inhibitor of the enzyme. The moiety is preferably a metal ion, metal ion complex comprising two or more metal ions or an organic cofactor.

Particularly preferred metal ion complexes are Cu₂ and Cu₃ containing complexes.

The protein used in the kit according to this second aspect of the invention may be an oxygen carrier, oxygenase enzyme, oxidase or hydrogenase. Suitable examples include hemocyanins, polyphenol oxidases (tyrosinases), multicopper oxidases like laccases, cytochrome P450 enzymes or Ni/Fe hydrogenases.

Preferably, the analyte is oxygen or hydrogen.

Preferably, the kit further comprises a reaction vessel, wherein the protein is contained within the reaction vessel. The protein may be in solution in a liquid medium or alternatively immobilised in a carrier within the reaction vessel. The medium suspected of containing the analyte is preferably added to the reaction vessel when the kit is used for detection of an analyte. In the preferred embodiment of the invention wherein the protein is an enzyme and the analyte is oxygen, the reaction vessel typically also comprises a substrate for the enzyme, in addition to oxygen.

Preferably, the protein in the kit according to the second aspect of the present invention is in contact with electrodes, most preferably immobilised onto the surface of electrodes.

The method may be performed in a kit comprising an optical set-up that makes use of total internal reflection to excite a layer of fluorescently labelled protein molecules immobilised on electrodes. The electrodes are mounted in an optical microscope equipped with laser excitation and a high aperture objective to monitor the fluorescence emitted from the protein coated on the electrode. The electrodes are transparent to light of wavelength for exciting the fluorescent protein residues and to the fluorescence emitted by the label. In addition, a three electrode electrochemical set-up may be connected to the sample compartment and the electrode immersed in buffer to which enzyme substrate can be added. The enzyme may be regenerated either by a voltage sweep or chemically by making the electrode part of the flow cell and directing a redox active flow over the electrode.

In accordance with a third aspect of the invention we provide a protein comprising a moiety capable of binding an analyte and a fluorescent label, wherein the protein is excitable to induce intrinsic emission therefrom, and the intrinsic emission is convertible through Fluorescence Resonance Energy Transfer (FRET) into emission from the fluorescent label and the amount of said FRET is affected by binding of the analyte to the moiety, and the binding of the analyte to the moiety does not induce a substantial conformational change in the protein.

The protein is suitable for use in the method and kit according to the first and second aspects of this invention and all of the preferred features with regard the protein, moiety and fluorescent label outlined above for these first and second aspects of the invention are applicable to this third aspect of the invention.

The protein may be used in a biosensor to monitor its activity with a greater sensitivity than in conventional methods. Experiments in the lower picomolar range are within reach, which opens up opportunities for investigating molecules which are only available in minute quantities. The method presented here has the potential to study analyte binding in enzymes and proteins at the single-molecule level. This greater sensitivity leads to specific advantages: almost unlimited miniaturization, applicability to much lower concentrations (sub-nanomol/L) and strongly enhanced specificity due to the absence of interference. The proposed method has great potential for application in high-throughput screening and in nanotech-based bioelectronics.

The invention will now be illustrated by reference to the following figures, in which

FIG. 1 illustrates the principle of the FRET based O₂ sensing;

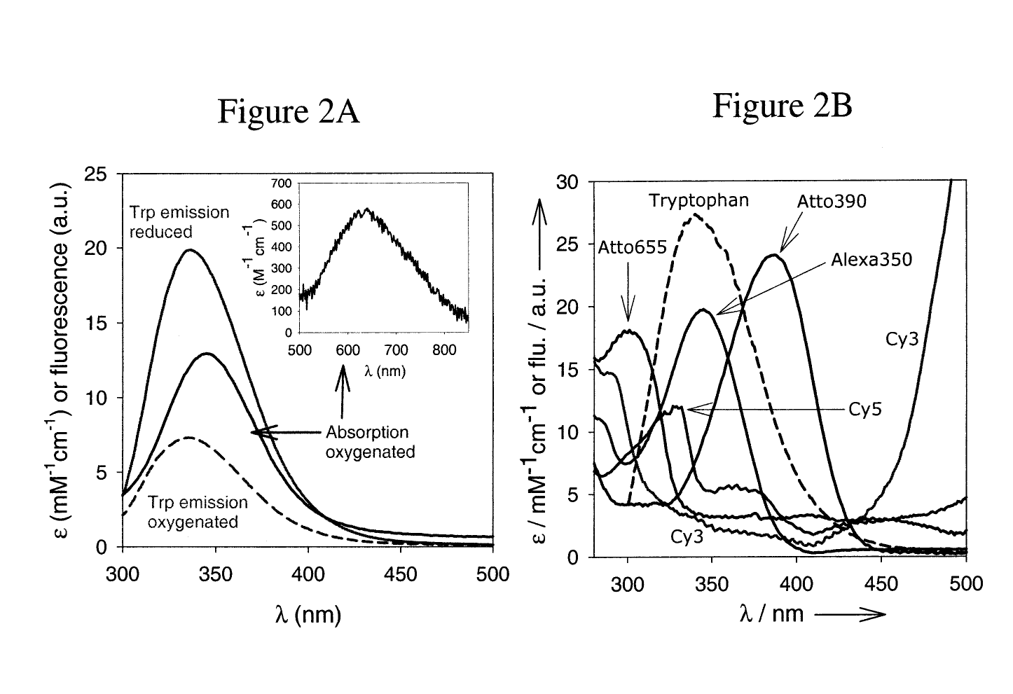

FIG. 2(A) shows the absorption spectrum of oxygenated Ty corrected for the contribution of the reduced protein between 300 and 500 nm (main panel) and between 500 and 850 nm (inset), and fluorescence emission spectra of fully reduced Ty and fully oxygenated Ty (dashed line) upon excitation at 280 nm;

FIG. 2(B) is the absorption spectra of the dyes Alexa350, Atto390, Cy3, Cy5 and Atto655 and their overlap with the Ty tryptophan emission (dashed line);

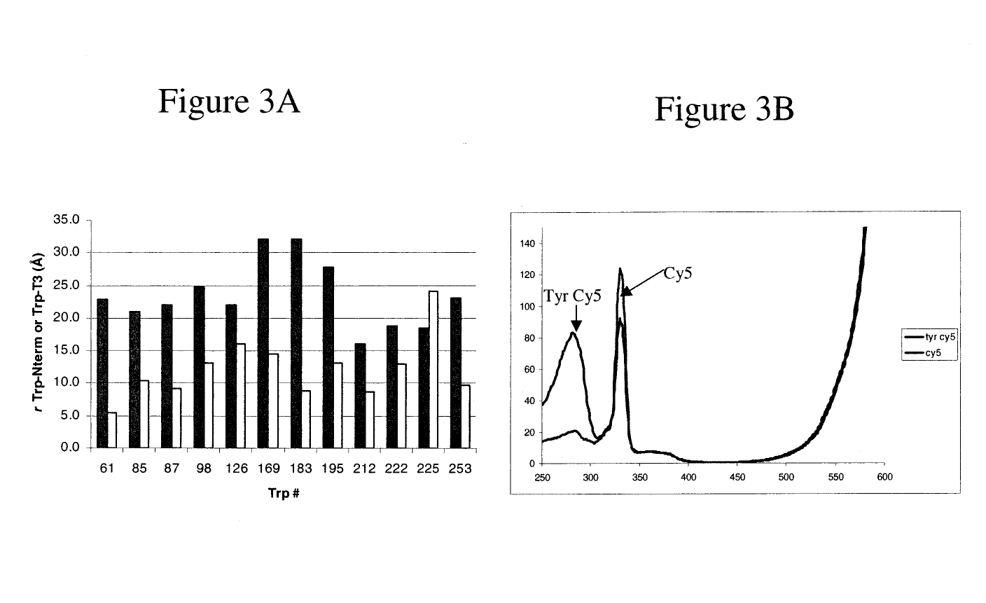

FIG. 3A shows the distances between Trp and the terminal-N (label attachment point; dark panels) and between Trp and the closest Cu of the type-3 center (light panels) in S. antibioticus Ty as modelled on the S. Castaneoglobisporus structure [7];

FIG. 3B shows the excitation spectrum of Ty-Cy5 and the free Cy5 dye at a detection wavelength of 665 nm;

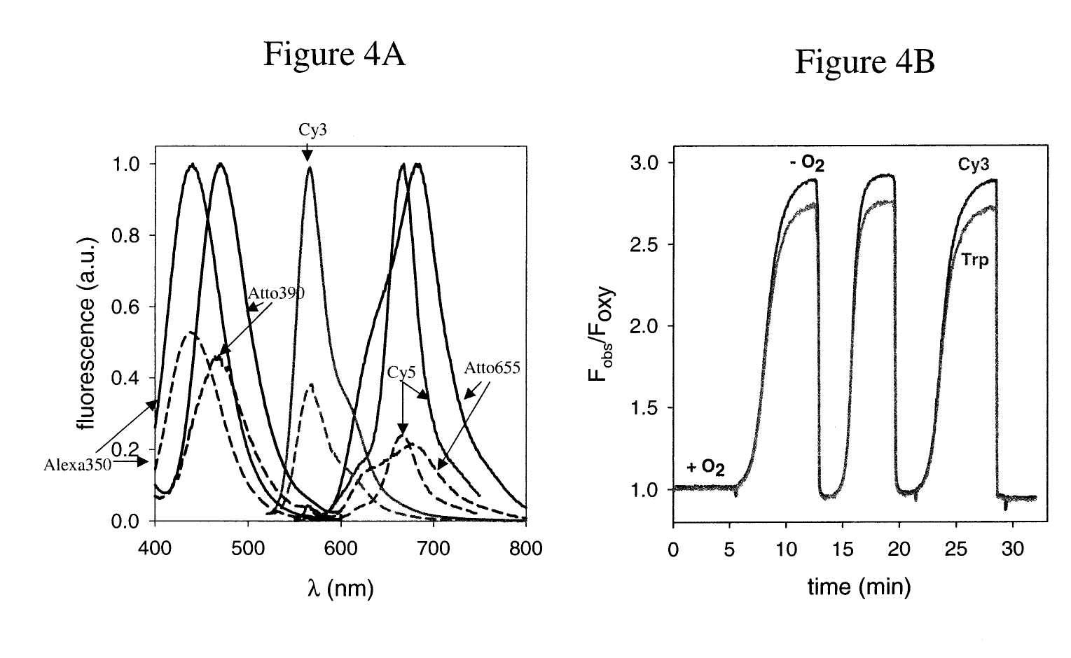

FIG. 4A shows the label emission of different Ty-label conjugates of the oxygen-free, reduced protein (solid lines) and of the oxygenated form (dashed lines) upon excitation at 280 nm;

FIG. 4(B) shows the reversible oxygenation and deoxygenation of a solution containing a mixture of unlabeled Ty (95%) and Ty conjugated with Cy3 (5%);

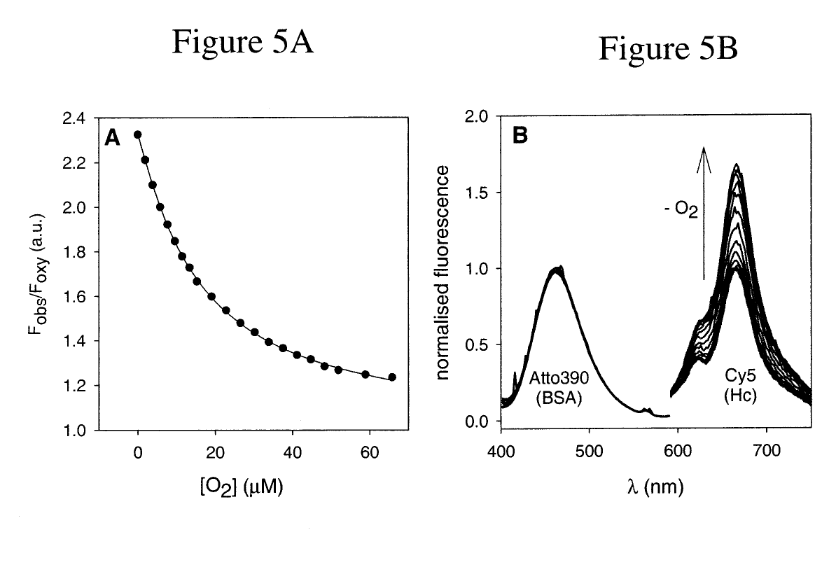

FIG. 5(A) shows the titration results of Hc-Atto390 with O₂ monitored by the dye emission upon excitation at 280 nm;

FIG. 5B shows the emission spectra (λₑₓ=280 nm) recorded for a mixture of BSA-Atto390 and Hc-Cy5 containing various [O₂];

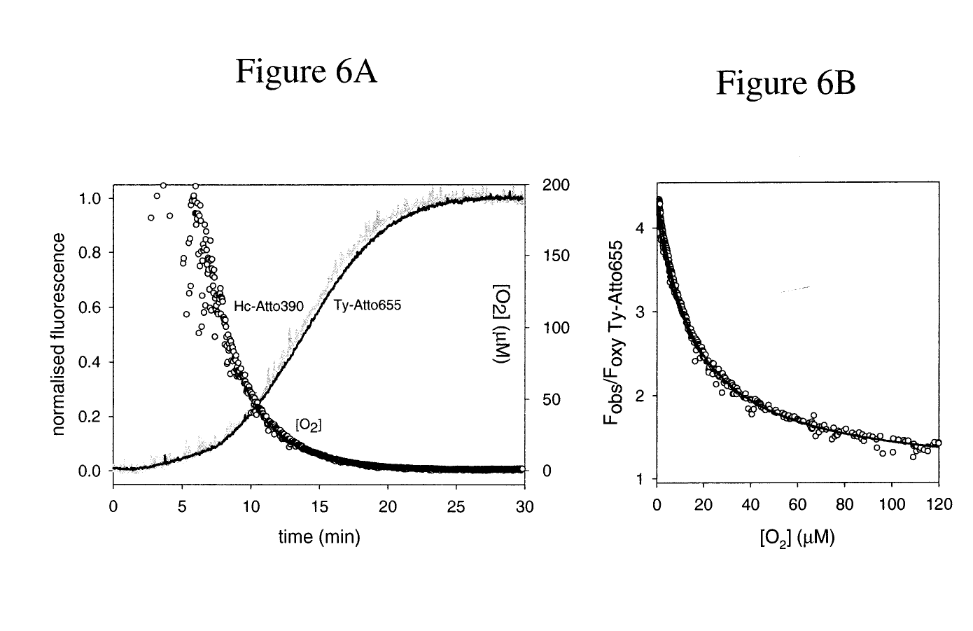

FIG. 6A shows the label fluorescence as a function of time for a mixture of tyrosinase-Atto655 and hemocyanin-Atto390 upon deoxygenation (initial [O₂]=˜0.6 mM) and excitation at 280 nm;

FIG. 6B shows the observed Ty-Atto655 fluorescence at each time-point in the time-trace (normalised to the intensity observed for the fully oxygenated protein) plotted against the calculated [O₂]; and

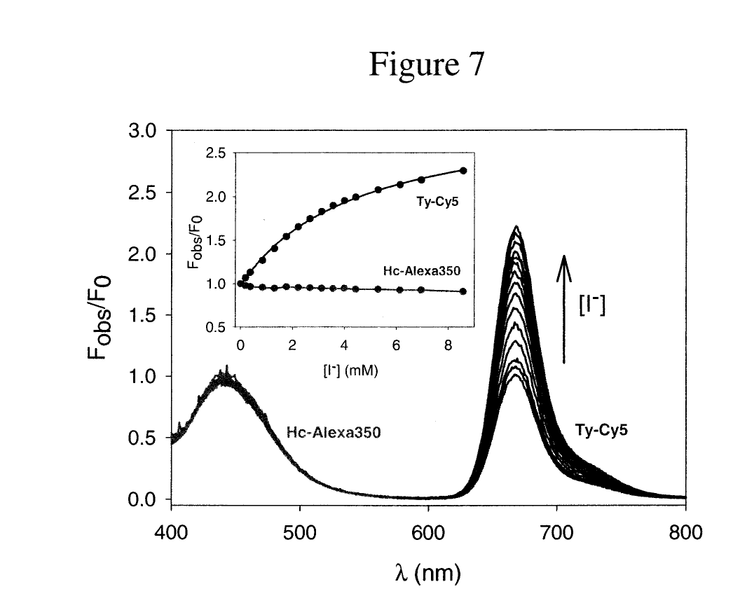

FIG. 7 illustrates the results of titration of a mixture of Hc-Alexa350 and Ty-Cy5 with iodide monitored by following the emission between 400 and 800 nm upon excitation at 280 nm.

### EXAMPLES

### Materials and Methods

To illustrate the present invention two type-3 copper proteins have been selected: Hc from the arthropod Carcinus aestuarii (Mediterranean crab) and Ty from the soil bacterium Streptomyces antibioticus. The C.a. Hc consists of a mixture of hexamers and dodecamers, which are self-assembled from three different types of subunits [8]. The S.a. Ty is a monomeric protein with a molecular mass of ˜30 kDa. The structure of Ty from S. castaneoglobisporus has recently been solved [7]. S.a. Ty has 91% sequence similarity (82% indentity) with S.c. Ty and can be easily modelled on the S.c. structure. It is known that Hcs may exhibit cooperative O₂ binding, unless they are dissociated into monomeric units. Dissociation can be achieved by incubating the Hc at high pH and in the absence of divalent metal ions. Both conditions were met in our experiments and we will assume, therefore, that the Hc used in this study was present in the form of monomers. The data on O₂ binding (vide infra) confirms this assumption.

### Example 1

### Protein Trp Fluorescence and O₂ Binding (Reference)

S.c. Ty contains 12 Trp residues on a total of 271 amino-acids (4.4% against ˜1% on average [9]), which cluster around the type-3 centre. The Trp fluorescence shows a maximum at 339 nm upon excitation at 280 nm. In an air-saturated solution (0.26 mM O₂), practically all protein occurs in the oxygenated form (see FIG. 2A). Upon complete deoxygenation, the Trp fluorescence increases by a factor of 2.7 while the shape and position of the emission band remain unchanged. For the C.a. Hc samples, this factor, further referred to as the switching ratio SR (=F_{red}/F_{oxy}), amounts to 2.2.

### Example 2

### Dyes (Reference)

FIG. 2B shows absorption spectra between 250 and 500 nm of the five dyes selected for this study: Alexa350, Atto390, Cy3, Cy5 and Atto655, as well as their overlap with the Ty tryptophan emission. The excitation wavelength was 280 nm. Together these dyes emit at wavelengths spanning the whole visible spectrum (Table 1 and FIG. 2B). This allows the researcher to choose a dye which emits at a wavelength which does not interfere with other fluorescent systems in the sample.

TABLE 1
Switching ratios, spectral overlap integrals and Förster radii for
Trp and the dyes utilised in this study.

| | | | | Jₜᵣₚ Ty^{[b]} | Rₒ, Trp |
|---|---|---|---|---|---|
| | λₑₘ | SR^{[a]} | SR^{[a]} | (nm⁴M⁻¹cm⁻¹ | Ty^{[c]} |
| Dyes | (nm) | Tyr | Hc | ×10⁻¹³) | (Å) |
| Trp | 339 | 2.7 | 2.2 | 13.0 | 23 |
| Alexa350 | 440 | 1.8 | 2.4 | 16.5 | 24 |
| Atto390 | 470 | 2.2 | 2.1 | 21.1 | 25 |
| Cy3 | 566 | 2.8 | 2.2 | 5.9 | 20 |
| Cy5 | 665 | 4.2 | 2.3 | 5.8 | 20 |
| Atto655 | 684 | 4.3 | 2.1 | 7.2 | 21 |

^{[a]}denotes the dye emission switching ratio (F_{red}/F_{oxy}) observed with excitation at 280 nm.
^{[b]}Calculated spectral overlap integrals between the Trp emission and the absorption of the oxygenated protein (for Trp) or between the Trp emission and the absorption of the dye (for the labels).
^{[c]}Förster radii Rₒ were calculated using φ = 0.07, K² =⅔ and n = 1.4. [10]

For Ty the spectral overlap integrals of the dye absorption bands with the Trp fluorescence spectrum have been collected in Table 1 together with the corresponding R₀ values. The latter are all in the range of the distances between the Trp residues and the amino-terminus (16-32 Å), which is the attachment point of the label (FIG. 3A and Table 2). This confirms that for Ty FRET from Trp to the selected labels will be observable. The Trp emission spectrum of Hc is similar to that of Ty and yields similar overlap integrals and R₀ values (not shown).

TABLE 2
Distances between Trp and the terminal-N and between Trp and the
closest Cu of the type-3 centre in S. Antibioticus Ty.

| Trp | rTrp-Nₑᵣₘ | rTrp-T3 |
|---|---|---|
| residue# | (Å) | (Å) |
| 61 | 22.8 | 5.5 |
| 85 | 21.0 | 10.4 |
| 87 | 22.1 | 9.3 |
| 98 | 24.7 | 13.1 |
| 126 | 22.1 | 16.1 |
| 169 | 32.0 | 14.5 |
| 183 | 32.2 | 8.9 |
| 195 | 27.9 | 13.1 |
| 212 | 16.1 | 8.7 |
| 222 | 18.8 | 13.0 |
| 225 | 18.4 | 24.0 |
| 253 | 23.0 | 9.8 |
| average | 23.4 | 12.2 |

### Example 3

### O₂ Binding and Label Fluorescence

Excitation absorption spectrums were recorded in this Example using a monochromator in the detection path, that has its wavelength set at an emission band of the molecule to be studied, in this case 660 nm for Cy5. Excitation is achieved by using a white light source and employing a second monochromator between light source and sample. The wavelength of this monochromator was slowly scanned. When the wave length of the excitation light matched an absoprtion band of Cy5, the dye started to fluoresce (at 660 nm) and the emitted light was observed in the detection set-up. By recording the fluorescence intensity as a function of the wavelength of the exciting light, the absorption spectrum was obtained.

Both Hc and Ty were labelled at their N-terminus with each of the five dyes [11]. When setting the detection wavelength at the dye emission maximum, the excitation spectra of the labelled proteins exhibit a strong peak at 280 nm, i.e., the wavelength of the Trp absorption (see FIG. 3B for Ty-Cy5). The peak around 330 nm is a second-order artefact. In a solution containing dye and protein separately (not linked) this peak (280 nm) was missing. The second order artefact is due to higher orders of light being allowed through the monochromator. The artefact can be excluded by placing a cut-off filter in the detection path that allows through light only beyond a certain wavelength (for example 600 nM).

These observations confirm that in the labelled proteins FRET from the Trps to the label occurs. The detection limit of the weakly fluorescent conjugates carrying a Cy3, Cy5 or Atto655 label was around 1 nM using standard equipment. The spectra (FIG. 3B) have been normalised to the emission intensity of Cy5 (excitation at 645 nm; detection at 665 nm).

In all cases, an increase in label emission was observed after deoxygenating the sample (FIG. 4A). This process was fully reversible as illustrated in FIG. 4B for a sample containing a mixture of unlabeled Hc (95%) and Hc-Cy3 (5%). The Figure shows Trp and label emission intensities as a function of time while the sample is repeatedly deoxygenated and oxygenated. The Cy3 fluorescence is specific for the labelled protein, while the Trp emission derives mainly (>95%) from unlabelled Hc. Similar observations were made for other labels, and also when Ty was used instead of Hc. The Trp and the label emissions vary in a similar manner. Additionally, the presence of the label does not affect the K_{d} for O₂ binding of the proteins.

### Control

To rule out that the changes in dye fluorescence might be due to a specific quenching by molecular oxygen, a mixture of Atto390 labelled bovine serum albumin (BSA) and Hc-Cy5 was deoxygenated while recording emission spectra from 300 to 750 nm upon excitation at 280 nm. The applied labels emit at different wavelengths, therefore permitting the simultaneous observation of each individual conjugate. The emission of Hc-Cy5 displays a clear dependence on [O₂], while the emission of BSA-Atto390 does not (FIG. 5B). With the labels exchanged, the reverse was observed. From this it can be concluded that the variations in label fluorescence correlate with the binding of O₂ to the labelled hemocyanin. Again, the fluorescence of the isolated dyes does not show a dependence on [O₂]. Similar observations were made for Ty.

### Cooperative or Non-Cooperative Oxygen Binding of Labeled Hc

Hcs, in the native multimeric forms, display cooperative O₂ binding due to allosteric interactions between the constituent subunits of the Hc. A titration of Hc carrying the Atto390 label with O₂ monitored by following the label fluorescence (FIG. 5A) showed that this cooperativity is lost. The solid line in FIG. 5A represents the best fit to the data. The titration data were fitted to a modified form of the Hill equation:

$${F}_{[ {O}_{2} ]} = {F}_{red} - \frac{( {F}_{red} - {F}_{oxy} ) \cdot {[ {O}_{2} ]}^{n}}{{[ {O}_{2} ]}^{n} + {K}_{d}} \qquad ( Equation 1 )$$

This resulted in a dissociation constant K_{d} of 22 μM and a Hill constant n of 1.09, diagnostic of almost total loss of cooperativity. It is known that arthropod Hcs dissociate into the subunits at high pH and in the absence of divalent metal ions. Since both conditions were met during the labelling procedure, it is likely that our Hc samples contain a mixture of dissociated labelled subunits. The Ty, in contrast, is monomeric and also does not show cooperative O₂ binding in the native or labelled forms (vide infra).

### Discussion—Switching Ratios

A few nanomolars of construct could be easily detected with a standard fluorimeter. Next to a good sensitivity, a high contrast in label fluorescence between O₂-free and O₂-bound protein is crucial for practical sensing applications. For the Hc, the dye switching ratio SR (=F_{red}/F_{oxy}), is similar to the SR value for the Trp fluorescence of the native protein (Table 1). For Ty, the SR values seem to vary between the labels. A quantitative interpretation of the SR values is beyond the scope of this report and will be published elsewhere. We still point out that very high contrast may be achieved by considering carefully the relative positions and orientations of the label, the type-3 centre and the fluorescent Trp residues.

### Example 4

### Simultaneous Observation of Different O₂ Sensing Conjugates

The fact that the described method works for labels showing widely different emission wavelengths opens the possibility to observe two or more constructs in the same sample using a single excitation wavelength. FIG. 6A illustrates an experiment involving Hc-Atto390 and Ty-Atto655 in a sample where [O₂] was gradually lowered by deoxygenating the solution. The circles refer to the oxygen concentration (right scale) as calculated from the fluorescence of the Hc-Atto390. The left scale refers to the Ty-Atto655 fluorescence. The experimental curves shown (left panel, left axis) have been normalized to the start and end values to facilitate comparison. The raw data of the Hc-Atto390 traces were used to calculate [O₂] at each time point (left panel, open circles, right axis) using Eq. 1 with K_{d}=22 μM and n=1.09 as determined through a direct O₂ titration of Hc-Atto390. The O₂ dissociation rate is in the range of 2-30 s⁻¹ for the Hc [12] and is ˜300 s⁻¹ for the Ty [13], meaning that the proteins can be considered to be in full equilibrium with the O₂ in solution on the timescale of the experiment.

The [O₂] at each time-point in the trace was determined using the response of Hc-Atto390 fluorescence with Eq. 1 and the oxygen dissociation constant and the Hill coefficient determined in an independent O₂ titration (FIG. 6A). The Ty-Atto655 fluorescence was then plotted as a function of the determined [O₂] at each time-point (FIG. 6B). The resulting Ty-Cy5 O₂ binding curve could be accurately fitted to the Hill equation, yielding a K_{d} of 15 μM (n=1.0), which is within error of the literature dissociation constant of 16 μM [14]. The experiment illustrates the accuracy of the [O₂] determination by monitoring the Hc-Atto390 fluorescence.

### Example 5

The specificity of the method is illustrated by the data presented in FIG. 7, which show a titration of a mixture of Hc-Alexa350 and Ty-Cy5 with iodide in an air-saturated solution. Iodide is known to displace oxygen from the Ty active site [15], while this does not occur for C.a. Hc [16]. The difference in iodide binding between Hc and Ty is reflected in the behaviour of the dye fluorescence upon increasing the [I⁻]: an increase in label fluorescence is observed for Ty-Cy5, while the Hc-Alexa350 fluorescence changes minimally. The small fluorescence decrease of the latter can be ascribed to the aspecific collisional quenching of the label and/or the Trp fluorescence by iodide [9]. The Ty-Cy5 fluorescence intensity vs. [I⁻] could be accurately fitted to Eq. 1, yielding an apparent K_{d} of 4.4±0.3 mM (n=1.00). The difference between the experimental K_{d} and the literature value of 3.0±0.3 mM [15] may be related to its dependence on the [O₂] in the sample, which may have varied between the two experiments.

### REFERENCES

[1]=Stryer et al (1967) Proc Natl Acad Sci USA 58, 179-726
[2]=Kenworthy et al (2001) Methods 24, 289-296
[3]=Tolosa et al (2002) Biotechnol. Bioeng 80, 594-597
[4]=Hartmann et al (1997) Sensors and Actuators B-Chemical 38, 110-115.
[5]=WO 2006/066977
[6]=Erker et al (2004) Biochem. Biophys. Res. Comm 324, 893-900
[7]=Matoba et al (2006) J. Biol. Chem 281, 8981-8990
[8]=Dainese et al (1998) European Journal of Biochemistry 256, 350-358
[9]=Lakowicz (1996) Principles of Fluorescence Spectroscopy, Kluwer Academic/Plenum Publishers, New York, Boston, Dordrecht, Moscow
[10]=Erker et al (2004) European Biophysics Journal with Biophysics Letters 33, 386-395
[11]=Kuznetsova et al (2006) Analytical Biochemistry 350, 52-60.
[12]=Messerschmidt et al (2001) Handbook of Metalloproteins, Volume 2
[13]=Hirota et al (2005) Journal of the American Chemical Society 127, 17966-17967.
[14]=Tepper et al (2004) J. Biol. Chem. 279, 13425-13434
[15]=Tepper et al (2002) J. Biol. Chem. 277, 30436-30444
[16]=Di Muro et al (2002) Z. Naturforsch 57, 1084-1091
[17]=Pickup et al (2005) Biosensors and Bioelectronics 20, 2555-2565

1. A method of detection of an analyte in which
(i) a protein comprising a moiety capable of binding the analyte and a fluorescent label is contacted with a medium suspected of containing the analyte;
(ii) the analyte, if present, binds to the moiety;
(iii) the protein is subjected to incident radiation to excite the protein and induce intrinsic emission therefrom;
whereby the intrinsic emission from the protein is converted through Fluorescence Resonance Energy Transfer (FRET) into emission from the fluorescent label and the amount of said FRET is affected by the binding of the analyte to the moiety; and,
(iv) the emission from the fluorescent label is measured;
whereby the level of emission from the fluorescent label is indicative of the presence of the analyte, and wherein the protein undergoes no substantial conformational charge during the method.

2. A method according to claim 1 wherein the binding of the analyte to the moiety reduces the amount of intrinsic emission from the protein that is converted through FRET into emission from the label.

3. A method according to claim 2 wherein the binding of the analyte enables the intrinsic emission from the protein to be converted through FRET to the moiety.

4. A method according to claim 1, wherein the level of emission from the fluorescent label is indicative of the concentration of analyte.

5. A method according to claim 1, wherein the intrinsic emission from the protein is from tryptophan residues or one or more organic cofactors in the protein.

6. A method according to claim 1, wherein the moiety is a metal ion, a metal ion complex comprising two or more metal ions or an organic cofactor.

7. A method according to claim 6 wherein the metal is a transition metal.

8. A method according to claim 7 wherein the transition metal is copper
or iron.

9. A method according to claim 7 wherein the transition metal is nickel.

10. A method according to claim 8 wherein the metal ion complex is Cu₂ or Cu₃.

11. A method according to claim 1, wherein the protein is an enzyme, preferably a redox enzyme.

12. A method according to claim 11 wherein the analyte is a (co-)substrate, cofactor or inhibitor of the enzyme.

13. A method according to claim 12 wherein the analyte is a (co-)substrate and is converted to another species by the enzyme during the method of detection.

14. A method according to claim 12 wherein the analyte is an inhibitor and binds reversibly to the moiety.

15. A method according to claim 11, wherein the enzyme is a monooxygenase enzyme or an oxidase.

16. A method according to claim 1, wherein the protein is a polyphenol oxidase, laccase or a cytochrome P450 enzyme.

17. A method according to claim 1, wherein the protein is an oxygen carrier, preferably hemocyanin.

18. A method according to claim 1, wherein the analyte is a gas under standard temperature and pressure, preferably oxygen.

19. A method according to claim 18 wherein the protein is a redox enzyme and catalyses the oxidation of a substrate using oxygen bound to the protein.

20. A method according to claim 19 further comprising a step of relating the emission from the fluorescent label to substrate turnover.

21. A method according to claim 18, wherein the medium is a biological sample and the emission from the label is indicative of metabolic rate.

22. A method according to claim 11, wherein the enzyme is a hydrogenase.

23. A method according to claim 22 wherein the analyte is hydrogen.

24. A method according to claim 1, wherein the fluorescent label absorbs radiation in the wavelength range 330-450 nm, preferably 350 nm, and fluoresces in the range 400-700 nm.

25. A method according to claim 24 wherein the fluorescent label is a dye selected from Cy5, Atto390, Alexa350 and Cy3.

26. A method according to claim 1, wherein the incident radiation has a wavelength in the range 260-450 nm, preferably 280-300 nm.

27. A method according to claim 1, wherein the fluorescent label is conjugated to a cysteine, lysine or arginine residue or the N-terminus of the protein, optionally through a linker.

28. A method according to claim 1, wherein the labelled protein is immobilised on a carrier.

29. A method according to claim 28 wherein the carrier is an electrode.

30. A method according to claim 28 wherein the carrier is microparticles of a sol-gel gas permeable matrix.

31. A method according to claim 1, wherein two or more proteins comprising a moiety capable of binding the analyte are contacted with a medium suspected of containing the analyte in step (i), and each moiety has a different binding affinity for the analyte.

32. A method according to claim 31, wherein the fluorescent labels on each
protein each fluoresce at a different wavelength.

33. A method according to claim 31, wherein the two or more proteins are enzymes and an allosteric effector is present in the medium suspected of containing the analyte.

34. A method according to claim 1, wherein the medium is suspected of containing two or more analytes and two or more proteins are contacted with the medium, and each protein comprises a moiety capable of binding one of the two or more analytes, and the level of emission from the fluorescent label on each protein is indicative of the presence of the analyte which binds the moiety of that protein.

35. A kit comprising a protein comprising a fluorescent label, an analyte, a radiation source for imposing incident radiation at a suitable wavelength for exciting the protein and inducing intrinsic emission therefrom, and a radiation detector capable of detecting the fluorescence emitted by the label, wherein the protein additionally comprises a moiety capable of binding the analyte and wherein the intrinsic emission from the protein may be converted through Fluorescence Resonance Energy Transfer (FRET) into emission from the fluorescent label, the amount of said FRET is affected by the binding of the analyte to the moiety, and wherein the binding of the analyte to the moiety does not induce a substantial conformational change in the protein.

36. A kit according to claim 35 wherein the radiation source has a wavelength of 260-450 nm, preferably 280-300 nm.

37. A kit according to claim 35 wherein the intrinsic emission from the protein is from tryptophan residues in the protein.

38. A kit according to claim 35, in which the moiety, protein or analyte is selected from the group consisting of:
a metal ion, a metal ion complex comprising two or more metal ions or an organic cofactor;
a transition metal;
an enzyme, preferably a redox enzyme;
a (co-)substrate, cofactor or inhibitor of the enzyme;
an analyte that is a (co-)substrate and is converted to another species by the enzyme during the method of detection;
an analyte that is an inhibitor and binds reversibly to the moiety;
a monooxygenase enzyme or an oxidase;
a polyphenol oxidase, laccase or a cytochrome P450 enzyme;
an oxygen carrier, preferably hemocyanin;
a gas under standard temperature and pressure, preferably oxygen;
a protein that is a redox enzyme and catalyses the oxidation of a substrate using oxygen bound to the protein;
a hydrogenase; and
hydrogen.

39. A kit according to claim 35, wherein the fluorescent label is selected from the group consisting of:
a fluorescent label that absorbs radiation in the wavelength range 330-450 nm, preferably 350 nm, and fluoresces in the range 400-700 nm;
a dye selected from Cy5, Atto390, Alexa350 and Cy3; and
a fluorescent label that is conjugated to a cysteine, lysine or arginine residue or the N-terminus of the protein, optionally through a linker.

40. A kit according to claim 35, further comprising a reaction vessel, wherein the reaction vessel comprises the protein and a liquid medium suspected of containing the analyte.

41. A kit according to claim 35, wherein the protein is an enzyme.

42. A kit according to claim 41 wherein the analyte is oxygen and the kit further comprises a (co-)substrate for the enzyme, in addition to oxygen.

43. A protein comprising a moiety capable of binding an analyte and a fluorescent label, wherein the protein is excitable to induce intrinsic emission therefrom, and the intrinsic emission is convertible through Fluorescence Resonance Energy Transfer (FRET) into emission from the fluorescent label and the amount of said FRET is affected by binding of the analyte to the moiety, and the binding of the analyte to the moiety does not induce a substantial conformational change in the protein.

44. A protein according to claim 43 in which the moiety or protein is selected from the group consisting of:
a metal ion, a metal ion complex comprising two or more metal ions or an organic cofactor;
a transition metal;
an enzyme, preferably a redox enzyme;
a (co-)substrate, cofactor or inhibitor of the enzyme;
an analyte that is a (co-)substrate and is converted to another species by the enzyme during the method of detection;
an analyte that is an inhibitor and binds reversibly to the moiety;
a monooxygenase enzyme or an oxidase;
a polyphenol oxidase, laccase or a cytochrome P450 enzyme;
an oxygen carrier, preferably hemocyanin;
a gas under standard temperature and pressure, preferably oxygen;
a protein that is a redox enzyme and catalyses the oxidation of a substrate using oxygen bound to the protein;
a hydrogenase; and
hydrogen.

45. A protein according to claim 43 wherein the fluorescent label is selected from the group consisting of:
a fluorescent label that absorbs radiation in the wavelength range 330-450 nm, preferably 350 nm, and fluoresces in the range 400-700 nm;
a dye selected from Cy5, Atto390, Alexa350 and Cy3; and
a fluorescent label that is conjugated to a cysteine, lysine or arginine residue or the N-terminus of the protein, optionally through a linker.
